(19) United States
(12) Patent Application Publication
(10) Pub. No.: US 2010/0196198 A1
(43) Pub. Date: Aug. 5, 2010

(54) OZONOE STERILIZAATION PROCESS AND APPARATUS

(75) Inventor: Bernard Legube, Chauvigny (FR)

Correspondence Address: ROBERT W. BECKER & ASSOCIATES, 707 HIGHWAY 333, SUITE B, TIJERAS, NM, 87059-7507, US

(73) Assignee: TS03 Inc. a corporation

(21) Appl. No.: 12/445,937

(22) PCT Filed: Oct. 18, 2007
(86) PCT No.: PCT/CA2007/001841

§ 371 Date: Apr. 16, 2009

Related U.S. Application Data
(60) Provisional application No. 60/829,977, filed on Oct. 18, 2006.

(51) Int. Cl. A61L 2/20

(52) U.S. Cl. 422/33

(57) ABSTRACT

A sterilization method is disclosed, which includes the steps of
providing a sterilization chamber;
placing the article into the sterilization chamber;
applying a vacuum of a preselected vacuum pressure to the sterilization chamber;
humidifying a sterilization atmosphere in the sterilization chamber;
maintaining the sterilization atmosphere at a temperature above 40° C. and at most 60° C.;
supplying ozone-containing gas to the sterilization chamber;
maintaining the sterilization chamber sealed for a preselected treatment period; and
releasing the vacuum in the sterilization chamber.

### FIELD OF THE INVENTION

The present invention relates generally to sterilization equipment and, particularly, to a method and apparatus for ozone sterilization.

### BACKGROUND OF THE INVENTION

Sterilization is the absolute destruction of any virus, bacteria, fungus or other micro-organism, whether in a vegetative or in a dormant spore state. Conventional sterile processing procedures for medical instruments involve high temperature (such as steam and dry heat units) or toxic chemicals (such as ethylene oxide gas, EtO). Steam pressure sterilization has been the time-honoured method of sterilization. It is fast and cost effective. However, the autoclave destroys heat-sensitive instruments. Thus, since more and more heat-sensitive instruments such as arthroscopes and endoscopes are used in medical treatment, other types of sterilization need to be used.

Ethylene oxide sterilization is used to cold sterilize heat-sensitive instruments. However, it has been deemed by national health and safety organizations to be carcinogenic and neurotoxic. Moreover, ethylene oxide requires long sterilization and aeration periods, since the molecule clings to the surface of instruments. This necessitates the use of containment rooms, monitoring systems, and room ventilators.

A more efficient, safer, and less expensive sterilization agent was needed and has been found in the form of ozone (O₃). Ozone can easily be generated from oxygen, especially hospital grade oxygen. Oxygen is readily available in the hospital environment, usually from a wall or ceiling oxygen source, or, if mobility is required, from a portable “J” cylinder of oxygen.

Ozone is widely used in industry as oxidizing agent to bleach paper pulp, treat drinking water, and sterilize sewage water and food products. Ozone generally acts on chemical compounds in two ways. Either by direct reaction or through hydroxyl radical species formed during the decomposition of ozone (Encyclopaedia Of Chemical Technology, Vol. 17, Ozone page 953 to 964). However, significant concentrations are required to make ozone gas an effective sterilant of micro-organisms. Furthermore, the high concentrations of ozone gas have to be combined with critical levels of humidity during the entire sterilization cycle to achieve reliable destruction of micro-organisms, in particular spores. The resistance of spores to ozone varies from strain to strain, but decreases with increasing relative humidity (Ishizaki et al., 1986. Inactivation of the Bacillus spores by gaseous ozone, J. Appl. Bacterial, 60:67-72). A high relative humidity is required for the ozone to penetrate through the protective shells of micro-organisms. A high relative humidity also permits ozone to penetrate the normally used sterilization packaging.

The use of a mixture of ozone gas with a very fine water mist in a sealed plastic bag container which contains an article to be sterilized is described in U.S. Pat. No. 3,719,017.

U.S. Pat. No. 5,069,880 describes a device capable of generating ozone at 85% humidity. Although ozone at this humidity can kill most micro-organisms, it may not meet the “worst case scenario” stipulated in North American standards.

In order to meet the standards imposed by the Food and Drug Administration and Health Canada, sterilizer manufacturers are required to achieve a minimum relative humidity level of 95%. Various prior patents (see Faddis et al., U.S. Pat. Nos. 5,266,275; 5,334,355; and 5,334,622) teach sterilization systems wherein water is heated to above the boiling point at ambient pressure to produce steam for injection into the ozone-containing gas produced by an ozone generator. The steam is heated to 120° C. Thus, the vapour/ozone mixture used for sterilization presumably has a temperature close to 100° C. However, since the decomposition of ozone increases exponentially with temperature in the range of 20 to 300° C., injecting the water vapour at a temperature of about 120° C. leads to premature ozone decomposition. Moreover, carrying out the sterilization at an elevated temperature and close to 100° C. will require a substantial cooling down period for the sterilized materials, thereby making the sterilization a lengthy and inefficient process.

A more efficient and effective sterilization method and apparatus is disclosed in WO 03/039607, which teaches a method for sterilization with ozone at a relative humidity above at least 95% and at a sterilization temperature of about 25-40° C. This temperature is chosen to maximize the half life of ozone during sterilization. Although excellent sterilization results are available with that method, meeting all existing standards, the sterilization cycle is very long. In order to guarantee a complete sterilization, cycle times of up to 4.5 hours are required. Thus, medical and dental services providers and hospitals used to make significant investments in multiple sets of equipment and tools to always have sterile equipment available.

There still exists a need for an environmentally and reliable sterilization process with shorter operating cycles.

### SUMMARY OF THE INVENTION

It is an object of the invention to provide a reliable and economical method and apparatus for the sterilization of an article with humidified ozone-containing gas, at shortened sterilization cycles.

It has been surprisingly discovered that significantly shortened sterilization cycle times can be achieved by operating the ozone sterilization with humidified ozone at an elevated temperature of 40 to 60° C. This is contrary to general knowledge which dictates sterilization temperatures as low as possible to avoid premature ozone decomposition. In view of the exponential increase in ozone decomposition with each increase in temperature, one would actually expect longer sterilization cycle times due to decreased ozone concentration at these elevated temperatures.

It is believed that the shortened sterilization periods are due to a marked increase in ozone susceptibility. It has been discovered that the increased ozone susceptibility far outweighs the increase in ozone decomposition.

The preferred sterilization method in accordance with the invention for the sterilization of an article includes the steps of:
providing a sterilization chamber;
placing the article into the sterilization chamber;
sealing the sterilization chamber;
applying a vacuum of a preselected vacuum pressure to the sterilization chamber;
maintaining the atmosphere in the sterilization chamber at a treatment temperature above 40 and at most 60° C.;
humidifying the sterilization chamber under vacuum, whereby the vacuum pressure is adjusted to maintain the boiling point of water in the sterilization chamber below the treatment temperature, and
injecting ozone into the sterilization chamber
maintaining the sterilization chamber sealed for a preselected treatment period;
releasing the vacuum in the sterilization chamber; and
removing the article from the sterilization chamber.

In a preferred embodiment, the temperature of the sterilization chamber atmosphere and the article is equalized after closing of the chamber to avoid localized condensation during the humidification step. Although equalization of the temperature of the article and the sterilization chamber can be achieved by simply waiting sufficiently long, this may result in an undesired delay of the sterilization procedure. Temperature equalization is preferably achieved by applying one or more equalization pulses wherein vacuum is applied to the chamber, followed by the injection of air or oxygen heated to the treatment temperature, or by re-circulating the air in the sterilization chamber after closing. This will result in the chamber, the article and the atmosphere in the chamber all being at the same temperature prior to commencement of the actual sterilization with ozone.

Preferably, heat is applied during the sterilization cycle to the chamber, a door of the chamber, any humidifier arrangement used for producing the water vapour and the water vapour piping to maintain them at the treatment temperature.

After release of the vacuum in the chamber, one or more ventilating cycles can be added to the preferred method for removing any remaining ozone and humidity from the sterilization chamber.

Accordingly, a sterilization apparatus in accordance with the invention includes
a sterilization chamber;
means for maintaining the temperature of the sterilization chamber, any materials placed therein, and an atmosphere in the sterilization chamber at a treatment temperature above 40 and at most 60° C.;
means for supplying ozone-containing gas to the sterilization chamber;
means for supplying water vapour to the sterilization chamber; and
means for applying a sufficient vacuum to the sterilization chamber to lower the boiling temperature of water below the treatment temperature.

Application of a sufficient vacuum to lower the boiling point of water to below the temperature in the sterilization chamber results in evaporation of any water in the chamber or in any space connected thereto. At the same time, all evaporated water present in the chamber or injected into the chamber is maintained in the vapour phase. Water vapour is preferably supplied to the chamber until saturation is reached. Water vapour is preferably generated in a humidifier arrangement which is also subjected to the vacuum applied to the sterilization chamber. The energy required for evaporation of the water is taken from the water itself and any components of the apparatus in contact with the water in the liquid phase. The result is a temperature drop in the humidifier, which may lead to a decrease in the evaporation rate. In the chamber the high relative humidity level combined with temperature differentials between walls and/or the load may lead to water condensation. Thus, the means for maintaining the treatment temperature are preferably means for heating at least one of the chamber, a chamber access door, the humidifier and the water vapour piping.

### BRIEF DESCRIPTION OF THE DRAWINGS

Embodiments of the present invention will now be described, by way of example only, with reference to the attached Figures, wherein:

FIG. 1 shows a schematic illustration of an apparatus in accordance with the invention;

FIG. 2 is a cross-section through a preferred ozone generator used in an apparatus in accordance with the invention;

FIG. 3 is a flow diagram of a preferred method in accordance with the invention;

FIG. 4 is a flow diagram of the electrical and control system preferably used in the apparatus of FIG. 1; and

FIG. 5 is a schematic illustration of the cooling unit of the apparatus in accordance with the invention.

### DETAILED DESCRIPTION

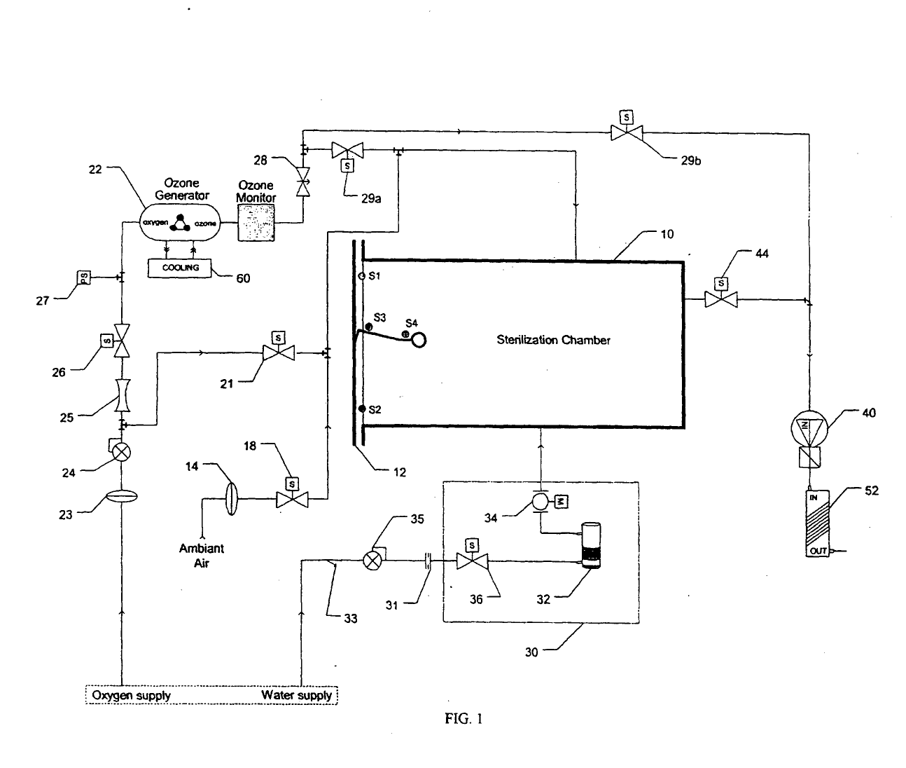

An ozone sterilizer in accordance with the invention as illustrated schematically in FIG. 1 operates in a relatively simple manner. Medical quality oxygen is subjected in an ozone generator 22 to an electrical field, which converts the oxygen into ozone containing gas. The ozone containing gas is then fed into a humidified sterilization chamber 10 where it sterilizes medical devices. The ozone is subsequently reconverted into oxygen using an ozone catalyst 52. The only residues left at the end of the sterilization cycle are oxygen and clean water vapour.

Single cycle sterilization with ozone is more efficient and provides for a shorter sterilization cycle than sterilization with EtO and requires few changes in user habits. Moreover, the ozone-based process in accordance with the invention is compatible for use with current packaging, such as sterile pouches and rigid containers. Moreover, the humidified ozone process of the invention provides significantly reduced sterilization cycle times due to an increase in ozone susceptibility.

This allows hospitals to reduce the cost of maintaining expensive medical device inventories. The ozone sterilization method of the invention offers several further advantages. It produces no toxic waste, does not require the handling of dangerous gas cylinders, and poses no threat to the environment or the user's health. Stainless-steel instruments and heat-sensitive instruments can be treated simultaneously, which for some users will obviate the need for two separate sterilizers.

Prior art ozone sterilization apparatus and methods exist in which the sterilization atmosphere is humidified to increase the efficiency of the ozone sterilization process. WO 03/039607 disclosed a process and apparatus for sterilization at a relative humidity above 80% and close to 100% at temperatures of 25-40° C. The disclosed process is very reliable and permits the achievement of sterilization levels which fully comply with all regulating standards. However, the sterilization cycle times required for the achievement of complete sterilization are 4.5 hours and more, which requires the users of the process to maintain large equipment inventories if a continuing supply of sterile equipment is desired.

The present invention is an improvement of that process. It has been surprisingly discovered that significantly reduced cycle times are achievable by simply raising the operating temperature of the process, the treatment temperature, to above 40 and at most 60° C., preferably 50-55° C. Cycle times are reduced to less than half, although raising the operating temperature results in an exponential increase in the ozone decomposition rate. The inventors of the present invention have now surprisingly discovered that the higher temperature also leads to an exponential increase in the ozone susceptibility of the micro-organisms to be neutralized. The exact reason for this phenomenon is not fully understood. However, it appears that micro-organism spore neutralization is the rate limiting factor of the sterilization process. Although even dormant spores can be oxidized by ozone, re-humidified spores are more susceptible to ozone and can be more quickly sterilized. It has now been found that the temperature at which the sterilization is carried out appears to be the rate controlling factor in the ozone susceptibility of spores. It is believed that this is due to an accelerated re-humidification of the spores at higher temperatures. Although this would favour a sterilization temperature as high as possible, the increased ozone decomposition at elevated temperatures would render the process very costly to operate and, once again increase cycle times. The inventors of the present invention have now found that an optimal compromise between increased spore susceptibility and increased ozone decomposition can be reached at an operating temperature above 40 and at most 60° C., preferably 50-55° C., for example at around 55° C. At temperatures below this range, spore susceptibility is too low resulting in long cycle times and at temperatures above this range the increased ozone decomposition rate renders the process uneconomical.

The preferred sterilization apparatus in accordance with the invention as illustrated schematically in FIG. 1 includes a sterilization chamber 10 which can be sealed to contain a vacuum. This is achieved with an access door 12, which can be selectively opened for access into the chamber and which seals the chamber in the closed condition. The apparatus further includes an ozone generator 22 for supplying ozone-containing gas to the sterilization chamber, a humidifier arrangement 30 for supplying water vapour to the sterilization chamber, and a vacuum pump 40 (ISP500-B or DVSL501-B, manufacturer Anest Iwata). The vacuum pump 40 is used for the application of a sufficient vacuum to the sterilization chamber 10 to increase the penetration of the sterilizing gas and to be able to generate water vapour at a temperature below the temperature inside the sterilization chamber. The vacuum pump 40 in the preferred embodiment is capable of producing a sufficient vacuum in the sterilization chamber to lower the boiling point of water in the chamber below the actual temperature of the atmosphere in the chamber. In the preferred apparatus, the vacuum pump is capable of producing a vacuum of 0.1 mbar. Ozone produced in the ozone generator 22 is destroyed in an ozone catalyst 52 to which ozone-containing gas is fed either after passage through the sterilization chamber 10 or directly from the ozone generator 22 through valve 29b (optional). The ozone catalyst 52 (DEST 25, manufacturer TSO₃) is connected in series after the vacuum pump 40 to prevent ozone gas escaping to ambient. The ozone decomposing material in the preferred catalyst 52 is carulite. For economic and practical reasons, it is preferred to use a catalyst for decomposition of the ozone in the sterilization gas exhausted from the sterilization chamber 10. The catalyst destroys ozone on contact and retransforms it into oxygen with a certain amount of heat being produced. Catalysts of this type and their manufacture are well known to the person skilled in the art of ozone generators and need not be described in detail herein. Furthermore, other means for destroying the ozone contained in the sterilization gas will be readily apparent to a person skilled in the art. For example, the gas can be heated for a preselected time to a temperature at which the ozone decomposition is accelerated, for example, to 300° C.

The humidifier arrangement 30 includes a humidifier chamber 32 (HUM 0.5, manufacturer TSO₃) sealed to ambient and connected to the sterilization chamber 10 through a conduit and a vapour intake valve 34. The humidifier chamber 32 is equipped with a level control to always ensure a sufficiently high water level (not shown). Water is directly supplied to the humidifier chamber 32 from a purified water supply (not illustrated). Water is supplied to the humidifier chamber 32 by way of a filter 33, a pressure regulator 35, an orifice 31 and input valve 36. The water vapour produced in the humidifier chamber 32 enters the sterilization chamber 10 by way of a vapour intake valve 34. The humidifier chamber is also preferably equipped with a heating device (not shown) that maintains the temperature of the water sufficiently high to achieve a higher water vapour evaporation rate.

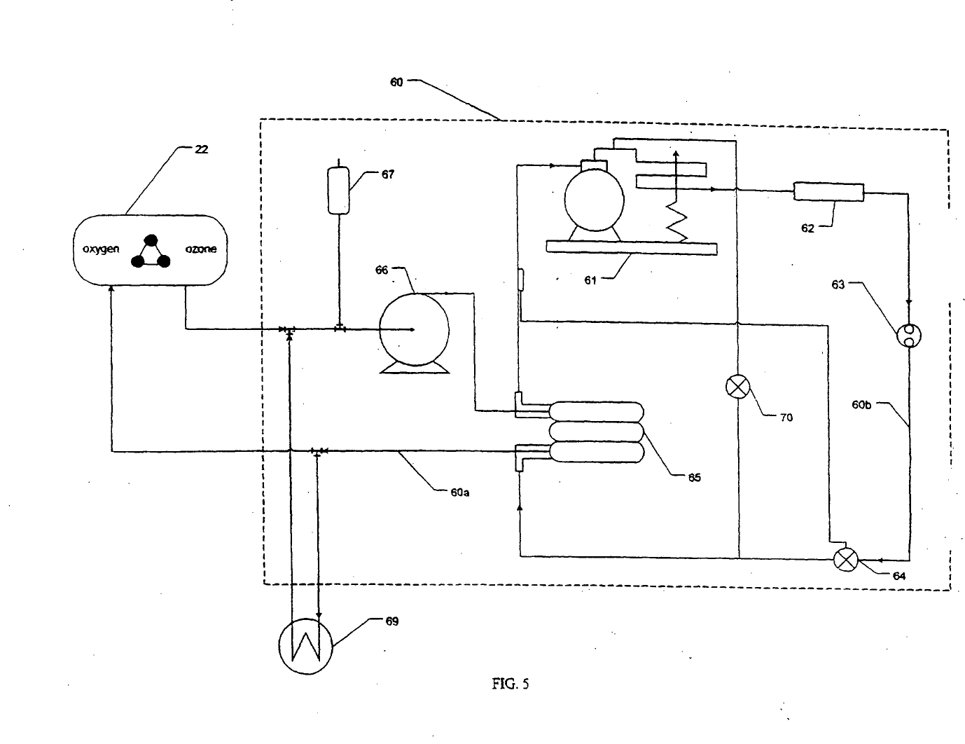

The ozone generator 22 (OZ, model 14a, manufacturer TSO₃) is of the corona discharge type and is cooled to decrease the ozone decomposition rate, all of which is well known in the art. The preferred generator produces ozone at a concentration of 120-122 milligram per liter most preferably 180 milligram per liter. To achieve a good lethality rate in an ozone sterilization process, the ozone supplied in the sterilization chamber should be sufficient to obtain a concentration of 20 to 85 milligram per liter preferably 40 to 45 milligram per litre. At these concentrations, the ozone generation is associated with a relatively high-energy loss in the form of heat. Generally, about 95% of the supplied electrical energy is converted into heat and only 5% is used to produce ozone. Since heat accelerates the inverse transformation of ozone into oxygen, it should be removed as quickly as possible by cooling of the ozone generator 22. The ozone generator in the apparatus is kept at the relatively low temperature of 4 to 6° C. by either an indirect cooling system 60 as illustrated in FIG. 5 with cooling water recirculation, or a direct cooling system with a refrigeration unit for cooling (not illustrated). The cooling system is preferably kept at the temperature of 4 to 6° C. In the preferred embodiment, the cooling system is kept at 4 to 6° C. so that the ozone-containing gas generated by generator 22 and entering into the sterilization chamber for sterilization is kept at a temperature of 50 to 55° C.

The ozone-generating unit 50 is preferably supplied with medical grade oxygen. The apparatus can be connected to a wall oxygen outlet common in hospitals or to an oxygen cylinder or to any other source capable of supplying the required quality and flow. The supply of oxygen to the generator 22 takes place across a filter 23, a pressure regulator 24, a flow meter 25 and oxygen shut off valve 26. The generator is protected against oxygen over pressure by a safety pressure switch 27. The ozone-oxygen mixture generated by the generator 22 is directed to the sterilization chamber 10 by a regulator valve 28 and a mixture supply solenoid valve 29a. The mixture can also be directly supplied to the ozone catalyst 52 by way of a bypass solenoid valve 29b (optional). In the preferred embodiment which includes a sterilization chamber of 125 liters volume, the pressure regulator 24 and the regulator valve 28 preferably controls the oxygen input at a pressure of about 116.5 kPa (2.2 psig) and a flow rate of about 1.5 liters per minute. However, it will be readily apparent to the skilled person that other flow rates may be used depending on the make and model of the ozone generator 22 and the size of the sterilization chamber.

The apparatus in accordance with the invention preferably includes a closed circuit cooling system using absolutely no fresh water (see FIG. 5). The cooling liquid flowing inside the generator 22 is a glycol-water mixture, which is cooled using R134a, an ozone layer friendly refrigerant. The cooling system is capable of maintaining the temperature between 3 and 6° C. The cooling system 60 of the generator 22 as shown in the schematic diagram of FIG. 5 includes a condensing unit 61 (Copelaweld M2FH-0049, manufacturer: Copeland), a drier 62 (UK-O53S, manufacturer: Alco), a sight glass 63 (optional) (ALM-1TT3, manufacturer: Alco), an expansion device 64 (Danfoss TUAE, orifice #4, manufacturer: Danfoss), an evaporator 65 (Packless FP3X812, manufacturer: FlatPlate), a hot gas bypass 70 (ADRI 1-1/4, manufacturer: Sporlan) a circulation pump 66 well known to the person skilled in the art, and an expansion reservoir 67. The cooling unit 60 is divided into a heat transfer circuit 60a and a refrigerating circuit 60b. The heat transfer circuit 60a includes the ozone generator 22, the coolant side of the evaporator 65, the circulation pump 66 and the expansion reservoir 67 (optional). The refrigeration circuit 60b includes the condensing unit 61, the drier 62, the sight glass 63, the expansion device 64, hot gas bypass 70 and the refrigerant side of the evaporator 65. The refrigerant circulating in the refrigeration circuit is R134a and the coolant flowing in the heat transfer circuit 60a is a glycol/water mixture.

The heat transfer circuit 60a can be omitted and the generator 22 included directly in the refrigeration circuit 60b. However, the use of an intermediate glycol/water filled heat transfer circuit is preferred, since the additional coolant acts as a larger heat sink so that energy peak loads generated upon activation of the generator 22 can be more reliably handled without significant swings in the temperature of the oxygen/ozone gas mixture produced.

The vacuum in the sterilization chamber 10 is produced by the vacuum pump 40 and the sterilization chamber drainage valve 44.

Valves 21, 26, and 36 are all the same (model: 6013A 5/32 FPM SS NPT1/4, manufacturer: Burkert). Valves 29a and 29b are Teflon solenoid valves (model: M442C1AFS-HT-1mic, manufacturer: Teccom). Valve 34 is preferably a solenoid valve which is the same model as the vacuum valve 44 (model: L9942302, manufacturer: Varian).

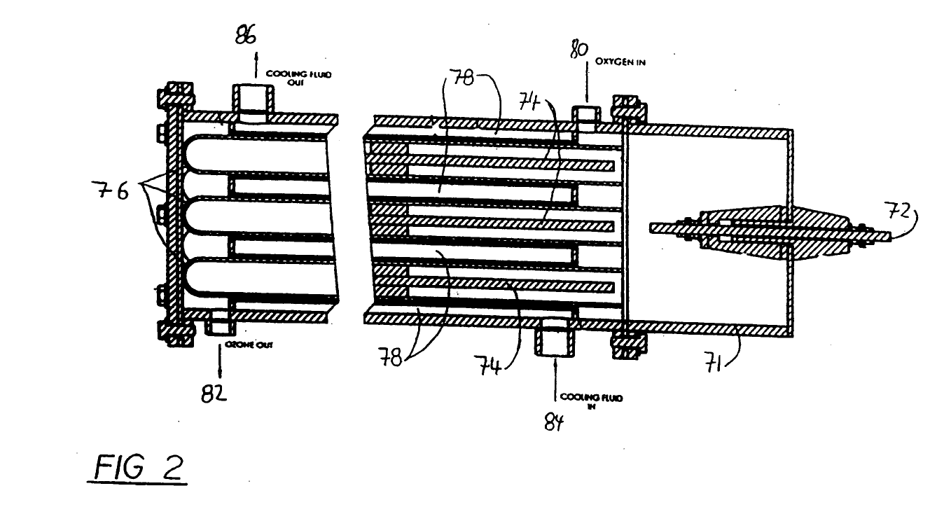

The preferred ozone generator used in the process and apparatus of the invention is schematically illustrated in FIG. 2 and is a generator of the corona discharge type well known to the person skilled in the art. The generator includes a first electrode 72 and a number of second electrodes 74 respectively centrally positioned in one of a corresponding number of reaction tubes 76. An ozone generating zone is defined between each second electrode 74 and the associated reaction tube 76. The electrodes are high voltage electrodes. Either electrode may be the ground electrode. The reaction tubes 76 are respectively surrounded by a cooling liquid channel 78 for cooling of the tubes. Oxygen enters the generator at an oxygen inlet 80 and ozone exits the generator at an ozone outlet 82. The reaction tubes are preferably made of a dielectric material, for example glass. The generator further includes an outer pressure vessel or housing 71 in which the oxygen inlet 80, ozone outlet 82 are provided as well as a cooling liquid inlet 84 and a cooling liquid outlet 86.

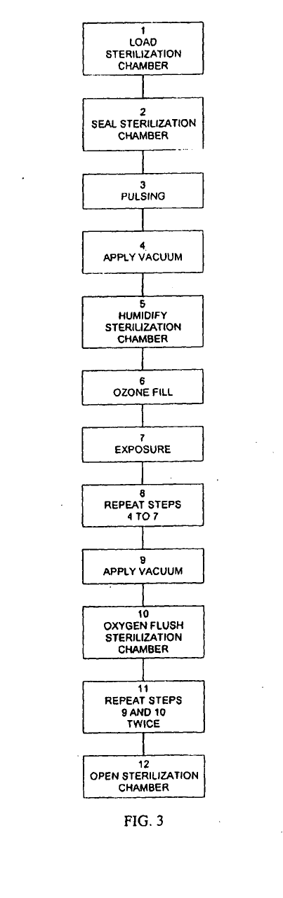

The preferred sterilization method according to the invention includes the following general steps as illustrated by the flow chart of FIG. 3. The medical instruments to be sterilized are sealed in sterile packaging containers or pouches such as generally used in the hospital environment and then placed into the sterilization chamber. The door of the sterilization chamber is closed and locked and the temperature equalization phase is started. This phase includes one or more pluses of ambient air or oxygen at ambient temperature through the sterilization chamber, or recirculation of the air in the chamber for a selected period of time. Then, vacuum is applied to the sterilization chamber. Water vapour is admitted into the sterilization chamber to humidify the chamber contents. A mixture of ozone and oxygen is supplied to the chamber and the chamber maintained sealed for a preselected treatment period. The vacuum application and ozone supply steps are preferably repeated at least once. To remove all remaining ozone in the sterilization chamber 10 after the sterilization cycle is completed a ventilation phase is commenced. After the ventilation phase the door is unlocked and the sterilized articles can be removed from the chamber. The temperature of the bottom and door of the chamber, of the water vapour piping and of the humidifier is preferably controlled throughout the sterilization process.

Before the sterilization cycle begins, the humidifier chamber 32 is filled with water to an adequate level. This is done by temporarily opening the water-input valve 36. Valve 36 also preferably opens automatically during the sterilization cycle if the water level is dropping below a preselected limit. Alternatively, water or water vapour can be injected directly into the sterilization chamber once a sufficiently low vacuum has been applied to evaporate all injected water and maintain it in the vapour phase.

If pulsed temperature equalization is used, air intake valve 18, oxygen supply valves 21 and 26, mixture supply valve 29a, and mixture bypass valve 29b are closed and vapour intake valve 34 and chamber drainage valve 44 are opened. The sterilization chamber 10 is evacuated to a vacuum pressure of about 330 mbar. Then the chamber drainage valve 44 is closed, intake valve 18 is opened and air is admitted into the chamber until ambient atmospheric pressure is reached. This sequence is repeated preferably 10 times, to ensure full temperature equalization. In the alternative, temperature equalization can also be carried out by recirculation of the evacuated chamber contents. This is achieved by redirecting the exhaust of the vacuum pump 40 back into the sterilization chamber for a selected period of time.

Thereafter, intake valve 18 is closed, chamber drainage valve 44 is opened and the sterilization chamber 10 is evacuated to a vacuum pressure of about 1.0 mbar. Water vapour inlet valve 34 is closed when the absolute pressure in the sterilization chamber falls below 60 mbar. Once a pressure of about 1.0 mbar is achieved, the chamber drainage valve 44 is closed and the vapour intake valve 34 opened to lower the pressure in the humidifier chamber 32 to the vacuum pressure in the sterilization chamber. That forces the water in the humidifier chamber to evaporate with the resulting water vapour automatically entering the sterilization chamber 10 due to the associated increase in volume. Preferably, during the humidification period, valve 34 opens and closes several times for a pre-set period of time to control the increasing rate of the relative humidity inside the chamber. Instead of using a humidifier chamber, humidity into the chamber could also be achieved with one or many spray nozzles connected to the water supply line. When valve 34 opens the pressure of the water flowing through the nozzle produces a water fog that evaporates into the volume under vacuum. Humidification is continued until a relative humidity of 75-100% is achieved. The preferred humidity level is 85-90%. Shortly before the end of the humidification period (usually about 2 to 6 min.), the ozone generator is activated. The flow of the oxygen/ozone mixture exiting the ozone generator is controlled at all times by regulator valve 28 capable of resisting the vacuum and of adjusting the flow to between 1 and 3 litres per minute. As an optional feature, the generator can be started at the same time as the humidification period begins. This is then achieved with supply valve 26 and mixture bypass valve 29b. Supply valve 26 opens to let oxygen enter the generator. The ozone-oxygen mixture produced by the generator is then guided directly into the ozone catalyst 52 through mixture bypass valve 29b. After a humidification period of 30 to 90 minutes, the oxygen-ozone mixture is guided into the sterilization chamber by opening the mixture supply valve 29a and closing the mixture bypass valve 29b. The oxygen-ozone mixture enters the chamber 10 until an ozone concentration of 85 milligram per liter in the chamber is achieved. The time required for this step is dependent on the flow rate and concentration of the ozone gas in the mixture (preferably 150 to 190 mg/l at NTP) and the ozone concentration can be monitored with equipment known in the art. The concentration of ozone in the sterilization chamber should be between 20 and 85 milligrams per liter, preferably 35-45 milligrams per liter. Once the desired concentration is reached, the mixture supply valve 29a is closed to seal off the sterilization chamber and to maintain the humidified ozone/oxygen gas mixture in the chamber under vacuum.

Once the sterilization chamber is filled with the sterilization gas (mixture of oxygen and ozone gas), the generator 22 is stopped, the oxygen supply valve 26 is closed, and the ozone is maintained in contact with the articles to be sterilized for up to about 20 minutes, for a sterilization chamber of a volume of 125 liters (4 cubic feet). At this stage, the sterilization chamber is still under the effect of a partial vacuum of about 610 mbar. In an optional second step, the pressure level is raised to about 900 mbar using oxygen as a filling gas. This pressure level is maintained for about 20 min. The cycle of applying a vacuum of about 1.0 mbar, injecting sterilization gas, humidifying and sterilization period, can be repeated, and the number of repeat cycles (mini cycles) selected to achieve complete sterilization of the instruments. To do so, the vacuum is reapplied after the sterilization period preferably at a pressure of about 1.0 mbar again. Once the vacuum reaches 1.0 mbar, the humidification phase is recommenced, followed by the renewed injection of an oxygen/ozone sterilization gas mixture, followed by the sterilization period. The number of repeat cycles needed in an experimental set-up of a method and apparatus in accordance with the invention including a 125 litre (4 cubic foot) chamber was 2. Each sterilization cycle included an ozone injection time of 10-40 minutes, preferably 20-25 minutes. This set-up conformed to the Security Assurance Level standards of the FDA (SAL 10-6).

To remove all remaining ozone and humidity in the sterilization chamber 10 after complete sterilization a ventilation phase is engaged. The ventilation phase begins after the last sterilization period. The chamber drainage valve 44 is opened and a vacuum is applied down to approximately 13.3 mbar. Vapour intake valve 34 closes when the pressure reaches 60 mbar to evacuate the remaining ozone in the humidifier. Once the vacuum pressure of 13.3 mbar is obtained, drainage valve 44 closes and the oxygen supply valve 21 opens, admitting oxygen into the sterilization chamber 10. Once atmospheric pressure is reached, the oxygen supply valve 21 is closed, the sterilization chamber drainage valve 44 is opened, and vacuum reapplied until a pressure of 6.6 mbar is reached. Finally, a last ventilation cycle, but this time down to 1.3 mbar, is done for a total of three ventilation cycles. Once atmospheric pressure is reached after the last cycle, the door mechanism of the sterilization chamber is activated to permit access to the contents of the sterilization chamber. The ventilation phase has two functions. First, to remove all ozone residues in the sterilization chamber before opening the access door and, second, to dry the sterilized material by evaporation when the vacuum pressure is applied. Of course, different vacuum pressures, cycle times and number of repetitions can be used, as long as the desired ozone removal and drying are achieved.

The ozone-containing gas evacuated from the sterilization chamber 10 is passed over the ozone catalyst 52 prior to exhausting the gas to the atmosphere to ensure a complete decomposition of the ozone in the sterilization gas. The ozone catalyst 52 is used during only two portions of the sterilization cycle, the activation of the generator 22 (with optional valves 26 and 29b) and the evacuation of the sterilization chamber 10. During the start up phase of the generator 22, the mixture bypass valve 29b is opened and the ozone is guided across the catalyst 52. Once the start-up phase of the generator 22 is complete, the bypass valve 29b closes. During evacuation of the sterilization chamber 10, the sterilization chamber drainage valve 44 is opened and the ozone containing sterilization waste gas is guided to the catalyst 52. Once the evacuation of the sterilization chamber 10 is completed, the drainage valve 44 is closed. The circulation of ozone is ensured by the vacuum pump 40. The ozone catalyst 52 can be located upstream or downstream of the vacuum pump 40.

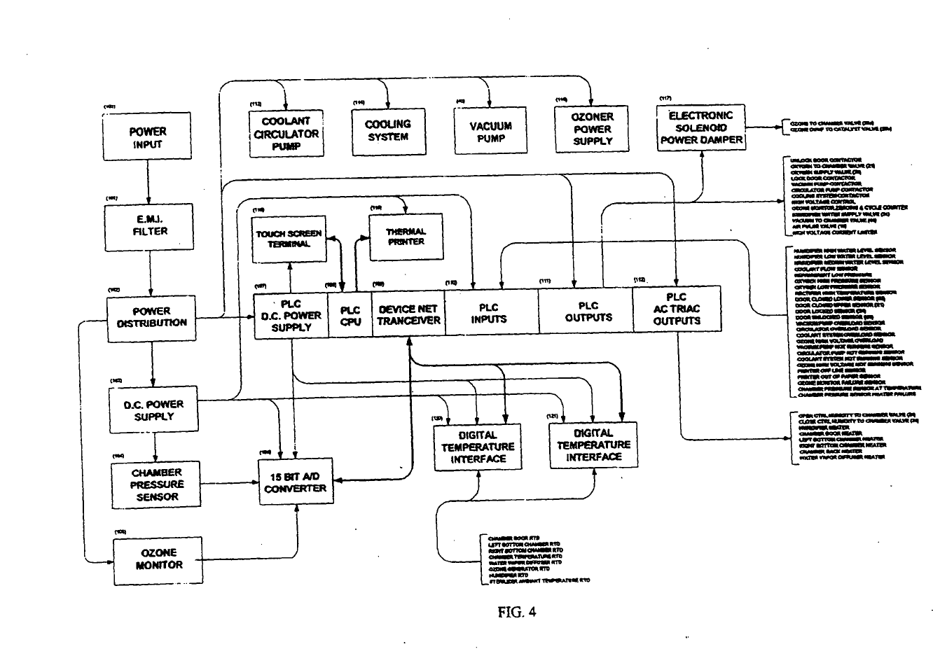

The sterilization apparatus is preferably controlled by the scheme presented in the electrical block diagram (FIG. 4 and Process Flow Diagram (FIG. 1). The control system is built around a PLC shelf (Programmable Logic Controller). This shelf contains a power supply (107) a CPU unit (108), a Device Net Transceiver (109), a 32×24 volts DC discrete input module (110), a 16×120 VAC discrete output module (111) and finally an 8×120 VAC TRIAC controlled output module (112). All those modules are disposed on a physical shelf that contains a data and address bus.

Device Net is an industrial serial communication protocol largely used in the industry for instrumentation and control. In this sterilization apparatus the Device Net transceiver (109) is used to communicate in full duplex, the data between the CPU (109) and the 15 bit A/D converter (106) and both Digital Temperature Interfaces (120), (121).

The PLC CPU possesses three RS232 ports. One is used to receive and send data to the Touch Screen Terminal (118), another one is used to send data to a thermal printer (119) and the last port is used as a service port where a PC (Personal Computer) can be hooked up to communicate with the PLC CPU (108) to load up the control protocol program. (Control Protocol Program is not in the scope of this document).

The Touch Screen terminal (118) is located at the front of the sterilizer beside the thermal printer (119). Touch Screen Terminal and thermal printer constitute a User Interface terminal.

Power needed for: “thermal printer (119), Device Net Link, (109), (106), (120), (121), Chamber Pressure Sensor (104) and PLC discrete inputs (111)” come from the DC Power Supply (103).

Chamber Pressure Sensor (104) and Ozone Monitor (105) have standard 0 to 10 VDC output signal. Both signals are sent to a 15 bits A/D converter. Then, both converted signals are sent to CPU by the Device net digital link for processing.

Power input (100) of the sterilizer is a four wire 208 VAC 3 phases in star configuration with neutral. The 3 phase power input is filtered to prevent conducted RFI (101). Then, power is distributed by power distribution buss (102) to the various electrical systems of the sterilizer apparatus.

A cooling system (60) is used to cool down the ozone generator. This system includes the cooling unit (114) and the coolant circulator pump (113). The temperature of the coolant in the generator is sense by an RTD located at the generator. The temperature is sent to the CPU (108) by the Device Net system (109) (120) (121). Coolant circulator (113) and cooling unit (114) are controlled by contactors driven by PLC outputs (111) which in turn are controlled of the software protocol. All input and output required to achieve cooling system control are listed on the electrical block diagram as: Circulator Pump Contactor, Cooling System Contactor, Circulator Overload Sensor, Cooling System Overload system, Coolant System Not Running Sensor, Circulator pump Not Running Sensor. Refrigerant Low Pressure and Coolant Flow Switch.

The vacuum control system includes the vacuum pump 40, a pressure switch (not illustrated) and a pressure sensor 104. The start and stop operations of the vacuum pump are controlled according to the control protocol. All input and output required for the vacuum system is listed on the diagram: Vacuum Pump Contactor, Vacuum Pump not running sensor, Vacuum pump Overload sensor, Vacuum to Chamber Valve (44), Air Pulse Valve (18) (when pulse temperature equalization is used) and Oxygen to Chamber Valve (21) The pressure sensor output is converted by the 15 bit ND converter (106) and sent to the CPU by the Device Net digital Link (109). The pressure sensor also possesses two discrete outputs indicating to the CPU (108) the following conditions: Chamber Pressure Sensor at Temperature and Chamber Pressure Sensor Heater failure. Those two signals are listed on the electrical block diagram as PLC inputs.

The sterilization chamber door actuator system includes an electric drive of the screw type and four inductive sensors which allow the detection of the presence of the door and the locked or unlocked position of the actuator as part of the control protocol. The door opening system is also used in the alarm conditions management protocol to assure the safety of the user. All input and output required to achieve the door actuator system are listed on the electrical block diagram as: Lock Door Contactor, Unlock Door Contactor, Door closed Lower Sensor (S2), Door closed Upper Sensor (S1), Door Locked Sensor (S4) and Door Unlocked sensor (S3).

The Ozone power supply (116) includes a full wave rectifier, an oscillator circuit and a high voltage transformer. The output of the transformer is hooked up to the ozone generator (22). The power supply (116) is mounted as a resonator using the non-ideal characteristics of the high voltage transformer. All of these components have been embedded in one enclosed enclosure which acts as a Faraday cage in order to prevent RFI spreading in the surrounding electronics. The PLC 108 controls the ozone production and ensures by way of the ozone monitor 104 that the concentration desired for sterilization is achieved and maintained throughout the sterilization cycle. All input and output required by the Ozone Generation System is listed on the diagram as: Electronic Oxygen Pressure Regulator Unit (26), Ozone to Chamber Valve (29a), Ozone Dump to Catalyst Valve (29b), Ozone Monitor Zeroing & Cycle counter, High Voltage Control, High Voltage Current Limiter, Temperature Sensor, Ozone High Voltage Not Running Sensor and Ozone monitor Failure Sensor. The Ozone Monitor is followed by a fixed size sapphire orifice. This orifice works in conjunction with the electronic oxygen pressure regulator to achieve constant flow regulation while ozone is injected in the chamber.

The control system is provided with a user interface 118. In the preferred embodiment, this interface includes a touch-sensitive liquid crystal display (LCD) screen 118, a printer 119 for performance reports and a communications port 153 (Series RS-232) allowing the user to receive and transmit information necessary for use of the apparatus. It will be readily apparent to the person skilled in the art that other types of user interfaces can be used such as touch-sensitive pads, keyboards, or the like, and other types of communications interfaces. Thermal printer status inputs appear on the electrical block diagram as: Printer Off Line Sensor and Printer Out of Paper.

The system in accordance with the invention is capable of producing a relative humidity level higher than 95%.

The energy needed to evaporate the water during the humidification phase is taken from many sources. It is taken principally from the water and the structure of the humidifier unit. This contributes to a further cooling of the humidifier, and its contents. In effect, at 20° C., water boils up to an absolute pressure of 23.3 mbar and at 35° C., water boils up to an absolute pressure of 56.3 mbar. The vacuum in the sterilization chamber is preferably adjusted at a pressure where the boiling temperature of water is lowered below the temperature in the sterilization chamber. That boiling temperature may be so low that the temperature of water inside the humidifier decreases rapidly. The evaporation process cools the humidifier to a point where room air moisture condenses This can be avoided in another preferred embodiment by heating the external surface of the humidifier sufficiently to keep the exterior of the humidifier unit and the water inside the humidifier chamber at room temperature. This is achieved with a heating arrangement (not illustrated) which will be readily apparent to the person of skill in the art. Also, because of the high level of relative humidity achieved inside the chamber there is condensation on chamber inner surfaces and inside water vapour piping. To reduce water condensation the bottom of the chamber, the door and the water vapour piping are also heated.

The water vapour generated in the humidifier unit increases the relative humidity in the sterilization chamber. The humidification phase is continued until the relative humidity of the gas surrounding the medical instruments contained in the packaging pouches and containers reaches a minimum of 75%, and, depending on the conditions, a minimum of 80 to 85% or even 95 to 100% may be preferred. For a sterilization chamber of an approximate volume of 125 liters, the water vapour admission increases the pressure to about 50 mbar in the sterilization chamber. This value is an approximation because it is temperature dependent.

Oxygen/ozone-containing sterilization gas is injected into the humidified sterilization chamber at a temperature close to ambient. The ozone-containing gas is not heated as in the prior art. For optimum operation of a sterilizer in accordance with the invention and having a 125 liters chamber, a system is preferably used which is capable of generating an ozone flow of about 1 to 3 litres per minute containing about 85 mg/l of ozone to obtain at least at total of 10600 mg of ozone for each of the fillings of the sterilization chamber.

In another preferred process, humidification of the sterilization chamber is carried out by a pair of atomizers. The water is supplied to each of the atomizers from a water tank hooked up to a purified water supply. Ozone is supplied to the atomizers from an ozone accumulation tank. The atomizers are made of ozone oxidation resistant material, and are installed directly in the sterilization chamber. When the vacuum level is reached in the sterilization chamber, the atomizers release water and ozone. The ozone is moistened inside the atomizer. The ozone/atomized water mixture penetrates the sterilization chamber. Injecting the water into the sterilization chamber under vacuum has the immediate effect of evaporating the water. The sterilization chamber operating temperature is 40 to 60° C., (preferably 50 to 55° C.), a temperature at which water evaporates at pressures of 73.8 to 199.4 mbar (123.5 to 157.6 mbar). Thus, the water becomes vapour due to the vacuum created by the vacuum pump. The resulting ozone/water vapour mixture penetrates the material to be sterilized.

The above-described embodiments of the present invention are intended to be examples only. Alterations, modifications and variations may be effected to the particular embodiments by those of skill in the art without departing from the scope of the invention, which is defined solely by the claims appended hereto.

1. A method for sterilizing en article in a sterilization gas atmosphere, comprising the steps of:
(a) providing a sterilization chamber;
(b) placing the article into the sterilization chamber;
(c) sealing the sterilization chamber;
(d) applying a vacuum to the sterilization chamber for adjusting the pressure in the sterilization chamber to a sterilization pressure lowering the boiling point of water in the sterilization chamber to a temperature below the temperature in the sterilization chamber;
(e) maintaining an atmosphere in the sterilization chamber at a treatment temperature of above 40° C. and at most 60° C.;
(f) humidifying the atmosphere in the sterilization chamber;
(g) supplying ozone-containing sterilization gas to the sterilization chamber;
(h) maintaining the sterilization pressure in the sterilization chamber for a preselected treatment period; and
(i) releasing the vacuum in the sterilization chamber.

2. The method of claim 1, further including the step of equalizing a temperature of the article, the atmosphere in the sterilization chamber and any components and materials in contact with the atmosphere, prior to humidifying the atmosphere.

3. The method of claim 1, when operated at a temperature in the sterilization chamber of 50 to 55° C.

4. The method of any one of claim 1, wherein the vacuum pressure is between 0.1 and 10 mbar.

5. The method of claim 4, wherein he vacuum pressure is between 0.5 and 2 mbar.

6. The method of claim 1, wherein the amount of water is selected to achieve a level of humidity in the (Original) sterilization chamber of 75% to 100%.

7. The method of claim 6, wherein the amount of water is selected to achieve a level of humidity of at least 85%.

8. The method of claim 1, wherein the steps (d) to (h) are repeated at least once.

9. The method of claim 8, wherein the steps (d) to (h) are repeated a number of times sufficient to ensure complete sterilization of the article.

10. The method of claim 1 further comprising the step of passing all gases evacuated from the sterilization chamber through a means for destroying ozone to prevent emission of ozone to the atmosphere.
